(19) United States
(12) Patent Application Publication
(10) Pub. No.: US 2016/0002673 A1
(43) Pub. Date: Jan. 7, 2016

(54) SOLAR STEAM PROCESSING OF BIOFUEL FEEDSTOCK AND SOLAR DISTILLATION OF BIOFUELS

(71) Applicant: William Marsh Rice University, Houston, TX (US)

(72) Inventors: Nancy J. Halas, Houston, TX (US); Peter Nordlander, Houston, TX (US); Oara Neumann, Houston, TX (US); Alexander Urban, Houston, TX (US)

(73) Assignee: WILLIAM MARSH RICE UNIVERSITY, Houston, TX (US)

(21) Appl. No.: 14/767,317

(22) PCT Filed: Feb. 18, 2014
(86) PCT No.: PCT/US2014/016845

§ 371 Date: Aug. 12, 2015

Related U.S. Application Data
(60) Provisional application No. 61/775,751, filed on Mar. 11, 2013.
(60) Provisional application No. 61/765,992, filed on Feb. 18, 2013.

(51) Int. Cl. C12P 7/10; C12M 1/34; C12M 1/02; C12M 1/42; C12M 1/107

(52) U.S. Cl. CPC C12P 7/10; C12M 35/02; C12M 23/36; C12M 27/00; C12M 41/12; C12M 41/40; C12M 41/00; C12P 2201/00

(57) ABSTRACT

A method of producing bioethanol that includes receiving a feedstock solution that includes polysaccharides in a vessel comprising a complex is described. The complex may be copper nanoparticles, copper oxide nanoparticles, nanoshells, nanorods, carbon moieties, encapsulated nanoshells, encapsulated nanoparticles, and/or branched nanostructures. The method also includes applying electromagnetic (EM) radiation to the complex such that the complex absorbs the EM radiation to generate heat. Using the heat generated by the complex, sugar molecules may be extracted from the polysaccharides in the feedstock solution, and fermented. Then, bioethanol may be extracted from the vessel.

### CROSS REFERENCE TO RELATED APPLICATIONS

This application claims priority under 35 U.S.C. §119(e) to U.S. Provisional Patent Application Ser. Nos. 61/765,992 and 61/775,751, which are incorporated by reference in their entirety.

### BACKGROUND

The production of bioethanol from cellulosic feedstock, such as switchgrass, may require that the feedstock be processed such that the fermentable carbohydrates and sugars are released from the initial material. This is an intensive processing step that currently requires the input of energy in the form of heat, the use of caustic chemicals to break down plant cell walls, or the use of enzymes for that purpose, all of which add cost to the final product, currently rendering it noncompetitive with respect to fossil-derived fuels.

### SUMMARY

In general, in one aspect, the invention relates to a method of producing bioethanol that includes receiving a feedstock solution that includes polysaccharides in a vessel comprising a complex. The complex may be copper nanoparticles, copper oxide nanoparticles, nanoshells, nanorods, carbon moieties, encapsulated nanoshells, encapsulated nanoparticles, and/or branched nanostructures. The method also includes applying electromagnetic (EM) radiation to the complex such that the complex absorbs the EM radiation to generate heat. Using the heat generated by the complex, sugar molecules may be extracted from the polysaccharides in the feedstock solution, and fermented. Then, bioethanol may be extracted from the vessel.

In general, in one aspect, the invention relates to a system for producing bioethanol that includes a vessel with a complex. The vessel is configured to receive a feedstock solution that includes polysaccharides in a vessel comprising a complex. The complex may be copper nanoparticles, copper oxide nanoparticles, nanoshells, nanorods, carbon moieties, encapsulated nanoshells, encapsulated nanoparticles, and/or branched nanostructures. The method also includes applying electromagnetic (EM) radiation to the complex such that the complex absorbs the EM radiation to generate heat. Using the heat generated by the complex, sugar molecules may be extracted from the polysaccharides in the feedstock solution, and fermented. Then, bioethanol may be extracted from the vessel.

### BRIEF DESCRIPTION OF DRAWINGS

FIG. 1 shows a schematic of a complex in accordance with one or more embodiments of the invention.

FIG. 2 shows a flow chart in accordance with one or more embodiments of the invention.

FIG. 3 shows a chart of the absorbance in accordance with one or more embodiments of the invention.

FIGS. 4A-4B show charts of an energy dispersive x-ray spectroscopy (EDS) measurement in accordance with one or more embodiments of the invention.

FIG. 5 shows a chart of the absorbance in accordance with one or more embodiments of the invention.

FIG. 6 shows a chart of an EDS measurement in accordance with one or more embodiments of the invention.

FIG. 7 shows a chart of the absorbance in accordance with one or more embodiments of the invention.

FIG. 8 shows a flow chart in accordance with one or more embodiments of the invention.

FIG. 9 shows a chart of the absorbance in accordance with one or more embodiments of the invention.

FIG. 10 shows a chart of an EDS measurement in accordance with one or more embodiments of the invention.

FIGS. 11A-11C show charts of the porosity of gold corral structures in accordance with one or more embodiments of the invention.

FIGS. 12A-12C show charts of the mass loss of water into steam in accordance with one or more embodiments of the invention.

FIGS. 13A-13B show charts of the energy capture efficiency in accordance with one or more embodiments of the invention.

FIG. 14 shows a system in accordance with one or more embodiments of the invention.

FIG. 15 shows a flowchart for a method of producing bioethanol in accordance with one or more embodiments of the invention.

FIG. 16 shows an example system for producing bioethanol in accordance with one or more embodiments of the invention.

FIG. 17 shows an example of a system in accordance with one or more embodiments of the invention.

FIG. 18 shows an example of a system in accordance with one or more embodiments of the invention.

FIGS. 19A and 19B show the temperature and pressure as a function of time in accordance with one or more embodiments of the invention.

FIG. 20 shows an amount of D-Glucose and D-Galactose extracted in accordance with one or more embodiments of the invention.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

In general, embodiments of the invention relate to the production of bioethanol using a steam generation process, where the steam is generated using nanoparticles.

The solar steam production using nanonparticles as described, for example, in U.S. application Ser. No. 13/326,482, U.S. patent application Ser. No. 13/514,762, and PCT Application No. US2011/062497, the contents of which are hereby incorporated by reference in their entirety, along with multiple applications thereof, may be used to process cellulosic feedstock either as steam or as a high pressure, high temperature liquid. In one or more embodiments of the invention, the application of solar steam may reduce the energy cost for processing the cellulosic feedstock. Moreover, the processing method for extraction of fermentable biomass may be also take advantage of this essentially free source of high temperature/high pressure steam using our earlier disclosed methods. The solar steam source may be adapted to provide both elevated temperatures and pressures as needed for a liquid water batch extractor.

In accordance with one or more embodiments of the invention, a second important processing step for biofuels, most specifically bioethanol, is distillation of the fermented extract to produce ethanol of the appropriate H₂O concentration, for a particular application, such as for use in vehicles. In one or more embodiments of the invention, nanoparticle-enabled solar steam generation may be performed on mixtures of liquids for distillation purposes. In these embodiments, the method may produce a richer ethanol distillate than normal thermal heating methods, which may simplify and streamline the final distillation step in bioethanol production.

In accordance with embodiments of the invention, bioethanol production from lignocellulosic biomass such as hay or straw may be used to generate fuel. Alfalfa and Coastal Bermudagrass are known non-food feeds. A pretreatment of the feedstock prior to fermentation is crucial in order to achieve a high ethanol yield. The cell walls of grass contain cellulose and hemicellulose. These polysaccharides need to be degraded into smaller units which are accessible to yeast to perform fermentation.

In one or more embodiments of the invention, nanoparticle-generated solar steam may be used for the hot water pretreatment of feedstock. If the solar steam is kept in a closed system, high temperature and a significant pressure builds up. The conditions that embodiments of the invention may reach are sufficient for the pretreatment process. A slurry of the feedstock in water is kept in a pretreatment module where the steam streams into so the temperature and pressure rises to a regime where the polysaccharides degrade into smaller units. This improves the fermentability of the raw material and will lead to higher ethanol yield.

Embodiments of the invention use complexes (e.g., nanoshells) that have absorbed EM radiation to produce the energy used to generate the heated fluid. The invention may provide for a complex mixed in a liquid solution, used to coat a wall of a vessel, integrated with a material of which a vessel is made, and/or otherwise suitably integrated with a vessel used to apply EM radiation to the complex. All the piping and associated fittings, pumps, valves, gauges, and other equipment described, used, or contemplated herein, either actually or as one of ordinary skill in the art would conceive, are made of materials resistant to the heat and/or fluid and/or vapor transported, transformed, pressurized, created, or otherwise handled within those materials.

A source of EM radiation may be any source capable of emitting energy at one or more wavelengths. For example, EM radiation may be any source that emits radiation in the ultraviolet, visible, and infrared regions of the electromagnetic spectrum. A source of EM radiation may be manmade or occur naturally. Examples of a source of EM radiation may include, but are not limited to, the sun, waste heat from an industrial process, and a light bulb. One or more concentrators may be used to intensify and/or concentrate the energy emitted by a source of EM radiation. Examples of a concentrator include, but are not limited to, lens(es), a parabolic trough(s), mirror(s), black paint, or any combination thereof.

Embodiments of this invention may be used in any residential, commercial, and/or industrial application where heating of a fluid may be needed. Examples of such applications include, but are not limited to, alcohol production (e.g., ethanol, methanol) as for a biofuels plant, chemical treatment, chemicals and allied products, (e.g., rubber, plastics, textile production), laboratories, perfumeries, air products (e.g., argon, hydrogen, oxygen), drug manufacturing, and alcoholic beverages.

In one or more embodiments, the complex may include one or more nanoparticle structures including, but not limited to, nanoshells, coated nanoshells, metal colloids, nanorods, branched or coral structures, and/or carbon moieties. In one or more embodiments, the complex may include a mixture of nanoparticle structures to absorb EM radiation. Specifically, the complex may be designed to maximize the absorption of the electromagnetic radiation emitted from the sun. Further, each complex may absorb EM radiation over a specific range of wavelengths.

In one or more embodiments, the complex may include metal nanoshells. A nanoshell is a substantially spherical dielectric core surrounded by a thin metallic shell. The plasmon resonance of a nanoshell may be determined by the size of the core relative to the thickness of the metallic shell. Nanoshells may be fabricated according to U.S. Pat. No. 6,685,986, hereby incorporated by reference in its entirety. The relative size of the dielectric core and metallic shell, as well as the optical properties of the core, shell, and medium, determines the plasmon resonance of a nanoshell. Accordingly, the overall size of the nanoshell is dependent on the absorption wavelength desired. Metal nanoshells may be designed to absorb or scatter light throughout the visible and infrared regions of the electromagnetic spectrum. For example, a plasmon resonance in the near infrared region of the spectrum (700 nm-900 nm) may have a substantially spherical silica core having a diameter between 90 nm-175 nm and a gold metallic layer between 4 nm-35 nm.

A complex may also include other core-shell structures, for example, a metallic core with one or more dielectric and/or metallic layers using the same or different metals. For example, a complex may include a gold or silver nanoparticle, spherical or rod-like, coated with a dielectric layer and further coated with another gold or silver layer. A complex may also include other core-shell structures, for example hollow metallic shell nanoparticles and/or multi-layer shells.

In one or more embodiments, a complex may include a nanoshell encapsulated with a dielectric or rare earth element oxide. For example, gold nanoshells may be coated with an additional shell layer made from silica, titanium or europium oxide.

In one embodiment of the invention, the complexes may be aggregated or otherwise combined to create aggregates. In such cases, the resulting aggregates may include complexes of the same type or complexes of different types.

In one embodiment of the invention, complexes of different types may be combined as aggregates, in solution, or embedded on substrate. By combining various types of complexes, a broad range of the EM spectrum may be absorbed.

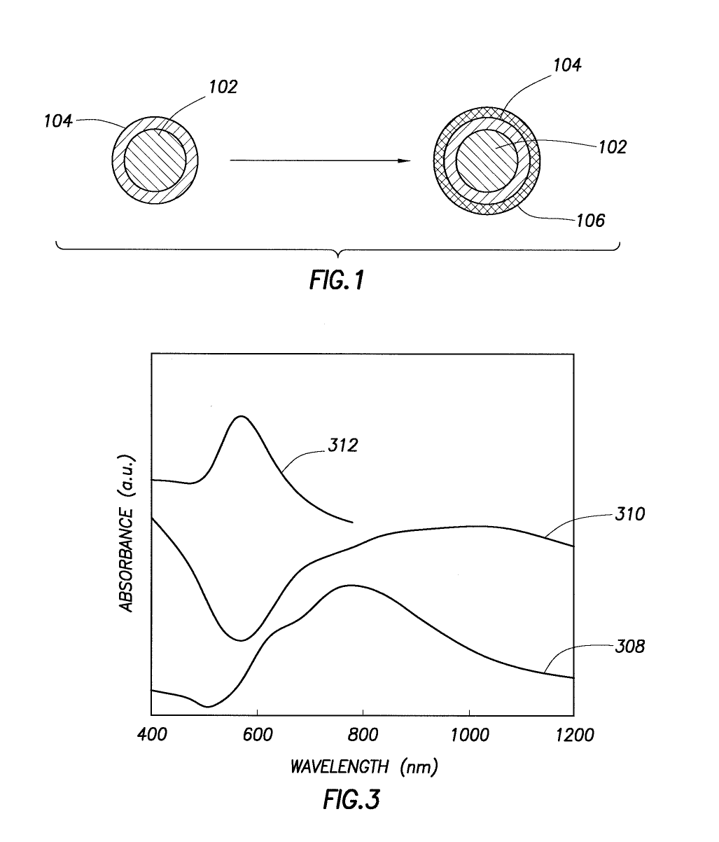

FIG. 1 is a schematic of a nanoshell coated with an additional rare earth element oxide in accordance with one or more embodiments of the invention. Typically, a gold nanoshell has a silica core 102 surrounded by a thin gold layer 104. As stated previously, the size of the gold layer is relative to the size of the core and determines the plasmon resonance of the particle. According to one or more embodiments of the invention, a nanoshell may then be coated with a dielectric or rare earth layer 106. The additional layer 106 may serve to preserve the resultant plasmon resonance and protect the particle from any temperature effects, for example, melting of the gold layer 104.

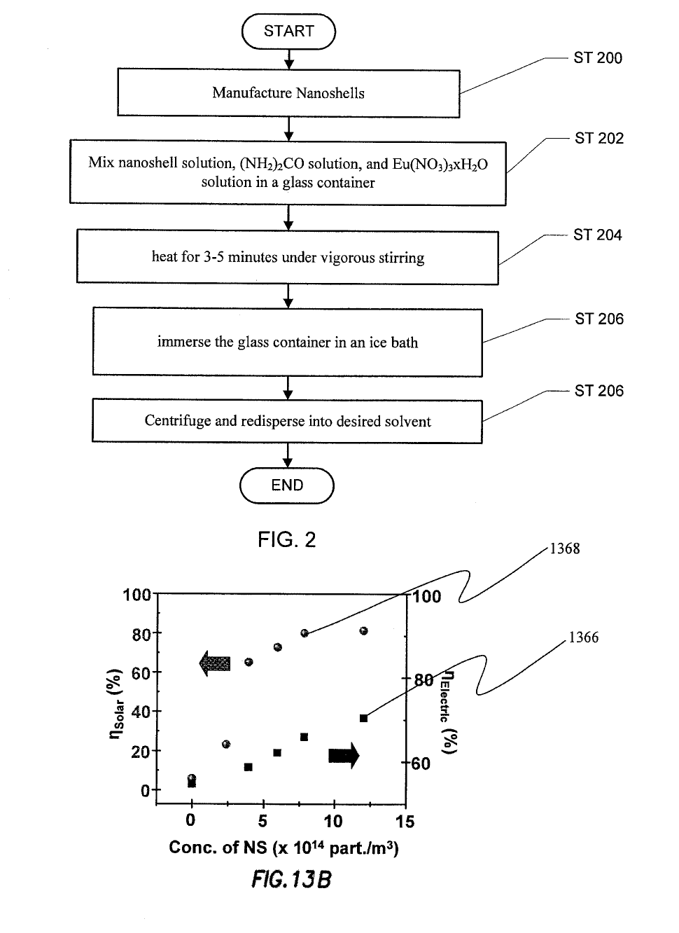

FIG. 2 is a flow chart of a method of manufacturing the coated nanoshells in accordance with one or more embodiments of the invention. In ST 200, nanoshells are manufactured according to known techniques. In the example of europium oxide, in ST 202, 20 mL of a nanoshell solution may be mixed with 10 mL of 2.5 M (NH₂)₂CO and 20 mL of 0.1 M of Eu(NO₃)₃xH₂O solutions in a glass container. In ST 204, the mixture may be heated to boiling for 3-5 minutes under vigorous stirring. The time the mixture is heated may determine the thickness of the additional layer, and may also determine the number of nanoparticle aggregates in solution. The formation of nanostructure aggregates is known to create additional plasmon resonances at wavelengths higher than the individual nanostructure that may contribute to the energy absorbed by the nanostructure for heat generation. In ST 206, the reaction may then be stopped by immersing the glass container in an ice bath. In ST 208, the solution may then be cleaned by centrifugation, and then redispersed into the desired solvent. The additional layer may contribute to the solubility of the nanoparticles in different solvents. Solvents that may be used in one or more embodiments of the invention include, but are not limited to, water, ammonia, ethylene glycol, and glycerin.

In addition to europium, other examples of element oxides that may be used in the above recipe include, but are not limited to, erbium, samarium, praseodymium, and dysprosium. The additional layer is not limited to rare earth oxides. Any coating of the particle that may result in a higher melting point, better solubility in a particular solvent, better deposition onto a particular substrate, and/or control over the number of aggregates or plasmon resonance of the particle may be used. Examples of the other coatings that may be used, but are not limited to silica, titanium dioxide, polymer-based coatings, additional layers formed by metals or metal alloys, and/or combinations of materials.

FIG. 3 is an absorbance spectrum of three nanoparticle structures that may be included in a complex in accordance with one or more embodiments disclosed herein. In FIG. 3, a gold nanoshell spectrum 308 may be engineered by selecting the core and shell dimensions to obtain a plasmon resonance peak at ˜800 nm FIG. 3 also includes a Eu₂O₃-encapsulated gold nanoshell spectrum 310, where the Eu₂O₃-encapsulated gold nanoshell is manufactured using the same nanoshells from the nanoshell spectrum 308. As may be seen in FIG. 3, there may be some particle aggregation in the addition of the europium oxide layer. However, the degree of particle aggregation may be controlled by varying the reaction time described above. FIG. 3 also includes a ˜100 nm diameter spherical gold colloid spectrum 312 that may be used to absorb electromagnetic radiation in a different region of the electromagnetic spectrum. In the specific examples of FIG. 3, the Eu₂O₃-encapsulated gold nanoshells may be mixed with the gold colloids to construct a complex that absorbs any EM radiation from 500 nm to greater than 1200 nm. The concentrations of the different nanoparticle structures may be manipulated to achieve the desired absorption of the complex.

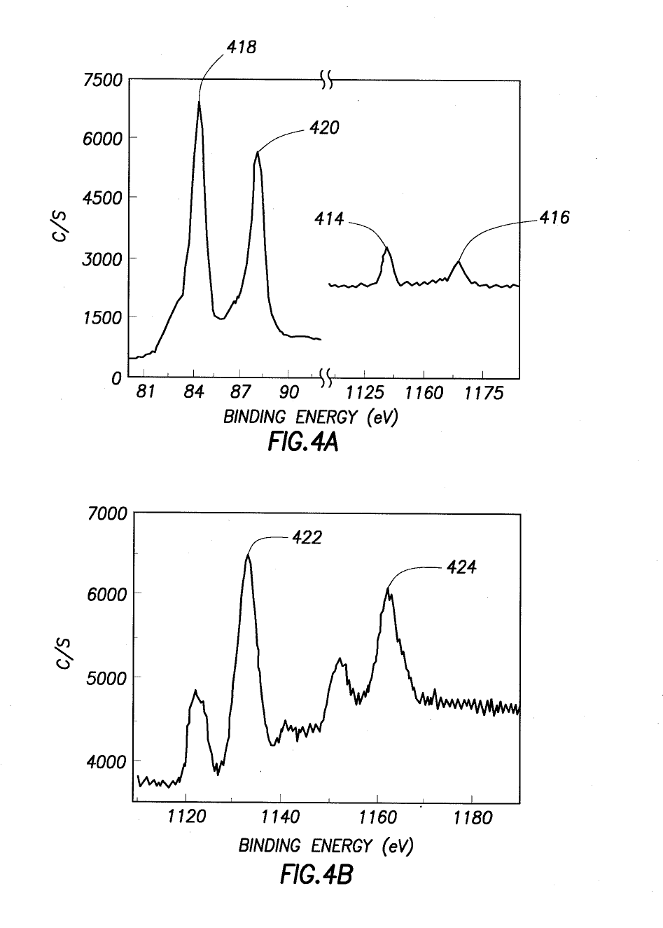

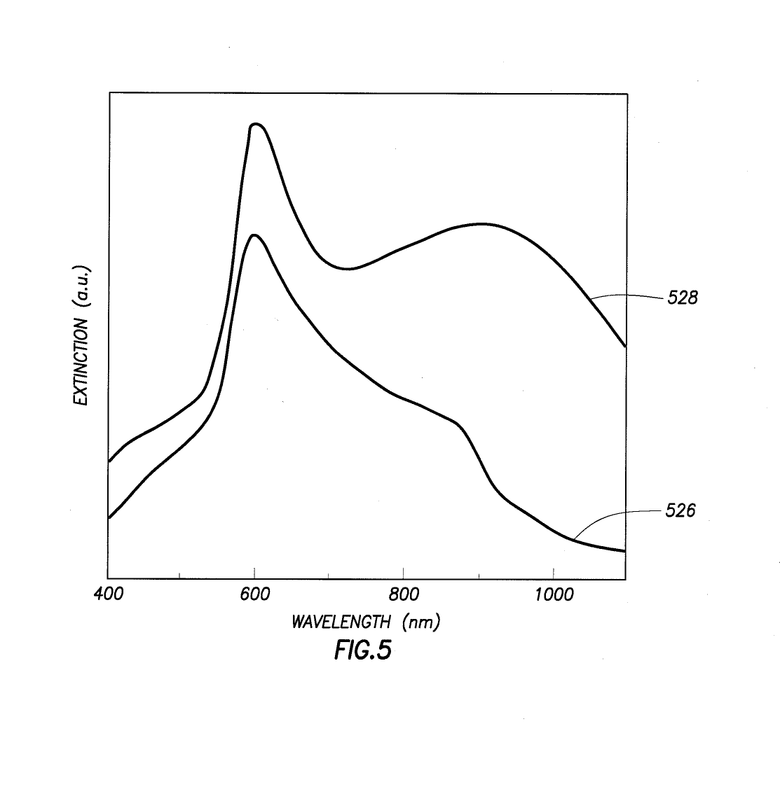

X-ray photoelectron spectroscopy (XPS) and/or energy dispersive x-ray spectroscopy (EDS) measurements may be used to investigate the chemical composition and purity of the nanoparticle structures in the complex. For example, FIG. 4A shows an XPS spectrum in accordance with one or more embodiments of the invention. XPS measurements were acquired with a PHI Quantera X-ray photoelectron spectrometer. FIG. 4A shows the XPS spectra in different spectral regions corresponding to the elements of the nanoshell encapsulated with europium oxide. FIG. 4A shows the XPS spectra display the binding energies for Eu (3d 5/2) at 1130 eV 414, Eu (2d 3/2) at 1160 eV 416, Au (4f 7/2) at 83.6 eV 418, and Au (4f 5/2) at 87.3 eV 420 of nanoshells encapsulated with europium oxide. For comparison, FIG. 4B shows an XPS spectrum of europium oxide colloids that may be manufactured according to methods known in the art. FIG. 4B shows the XPS spectra display the binding energies for Eu (3d 5/2) at 1130 eV 422 and Eu (2d 3/2) at 1160 eV 424 of europium oxide colloids.

In one or more embodiments of the invention, the complex may include solid metallic nanoparticles encapsulated with an additional layer as described above. For example, using the methods described above, solid metallic nanoparticles may be encapsulated using silica, titanium, europium, erbium, samarium, praseodymium, and dysprosium. Examples of solid metallic nanoparticles include, but are not limited to, spherical gold, silver, copper, or nickel nanoparticles or solid metallic nanorods. The specific metal may be chosen based on the plasmon resonance, or absorption, of the nanoparticle when encapsulated. The encapsulating elements may be chosen based on chemical compatibility, the encapsulating elements ability to increase the melting point of the encapsulated nanoparticle structure, and the collective plasmon resonance, or absorption, of a solution of the encapsulated nanostructure, or the plasmon resonance of the collection of encapsulated nanostructures when deposited on a substrate.

In one or more embodiments, the complex may also include copper colloids. Copper colloids may be synthesized using a solution-phase chemical reduction method. For example, 50 mL of 0.4 M aqueous solution of L-ascorbic acid, 0.8 M of Polyvinyl pyridine (PVP), and 0.01 M of copper (II) nitride may be mixed and heated to 70 degree Celsius until the solution color changes from a blue-green color to a red color. The color change indicates the formation of copper nanoparticles. FIG. 5 is an experimental and theoretical spectrum in accordance with one or more embodiments of the invention. FIG. 5 includes an experimental absorption spectrum 526 of copper colloids in accordance with one or more embodiments of the invention. Therefore, copper colloids may be used to absorb electromagnetic radiation in the 550 nm to 900 nm range.

FIG. 5 also includes a theoretical absorption spectrum 528 calculated using Mie scattering theory. In one or more embodiments, Mie scattering theory may be used to theoretically determine the absorbance of one or more nanoparticle structures to calculate and predict the overall absorbance of the complex. Thus, the complex may be designed to maximize the absorbance of solar electromagnetic radiation.

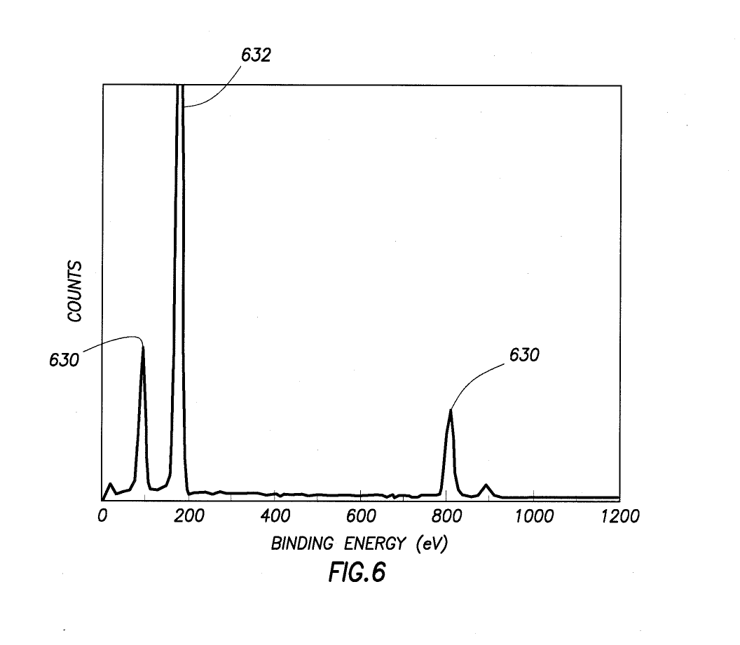

Referring to FIG. 6, an EDS spectrum of copper colloids in accordance with one or more embodiments of the invention is shown. The EDS spectrum of the copper colloids confirms the existence of copper atoms by the appearance peaks 630. During the EDS measurements, the particles are deposited on a silicon substrate, as evidenced by the presence of the silicon peak 632.

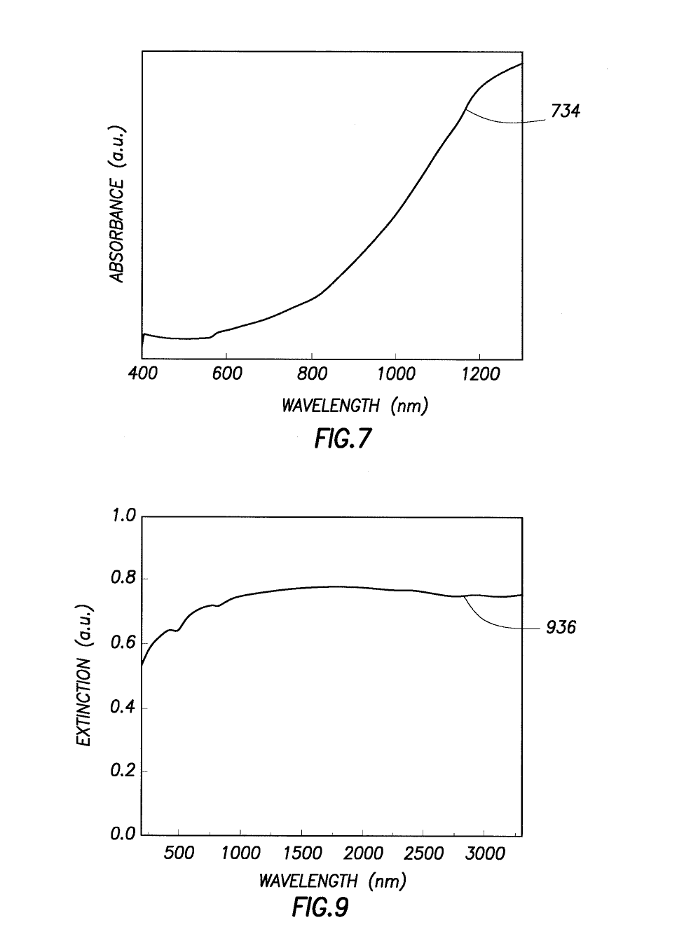

In one or more embodiments, the complex may include copper oxide nanoparticles. Copper oxide nanostructures may be synthesized by 20 mL aqueous solution of 62.5 mM Cu(NO₃)₂ being directly mixed with 12 mL NH₄OH under stirring. The mixture may be stirred vigorously at approximately 80° C. for 3 hours, then the temperature is reduced to 40° C. and the solution is stirred overnight. The solution color turns from blue to black color indicating the formation of the copper oxide nanostructure. The copper oxide nanostructures may then be washed and re-suspended in water via centrifugation. FIG. 7 shows the absorption of copper oxide nanoparticles in accordance with one or more embodiments of the invention. The absorption of the copper oxide nanoparticles 734 may be used to absorb electromagnetic radiation in the region from ˜900 nm to beyond 1200 nm.

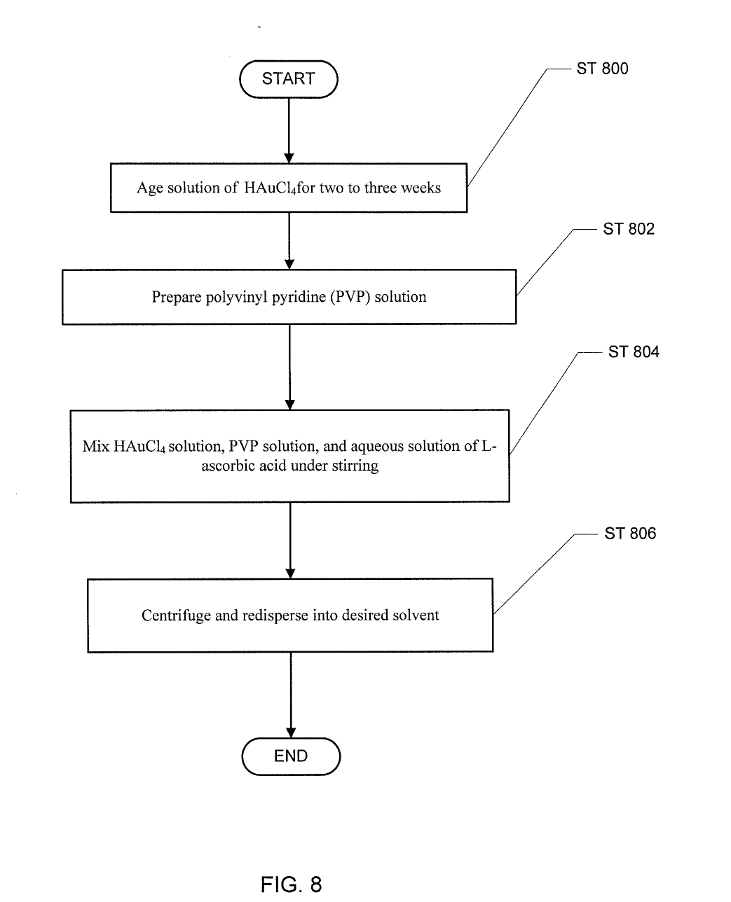

In one or more embodiments of the invention, the complex may include branched nanostructures. One of ordinary skill in the art will appreciate that embodiments of the invention are not limited to strict gold branched structures. For example, silver, nickel, copper, or platinum branched structures may also be used. FIG. 8 is a flow chart of the method of manufacturing gold branched structures in accordance with one or more embodiments of the invention. In ST 800, an aqueous solution of 1% HAuCl₄ may be aged for two-three weeks. In ST 802, a polyvinyl pyridine (PVP) solution may be prepared by dissolving 0.25 g in approximately 20 mL ethanol solution and resealed with water to a final volume of 50 mL. In ST 804, 50 mL of the 1% HAuCl₄ and 50 mL of the PVP solution may be directly mixed with 50 mL aqueous solution of 0.4 M L-ascorbic acid under stirring. The solution color may turn immediately in dark blue-black color which indicates the formation of a gold nanoflower or nano-coral. Then, in ST 806, the Au nanostructures may then be washed and resuspended in water via centrifugation. In other words, the gold branched nanostructures may be synthesized through L-ascorbic acid reduction of aqueous chloroaurate ions at room temperature with addition of PVP as the capping agent. The capping polymer PVP may stabilize the gold branched nanostructures by preventing them from aggregating. In addition, the gold branched nanostructures may form a porous polymer-type matrix.

FIG. 9 shows the absorption of a solution of gold branched nanostructures in accordance with one or more embodiments of the invention. As can be seen in FIG. 9, the absorption spectrum 936 of the gold branched nanostructures is almost flat for a large spectral range, which may lead to considerably high photon absorption. The breadth of the spectrum 936 of the gold branched nanostructures may be due to the structural diversity of the gold branched nanostructures or, in other works, the collective effects of which may come as an average of individual branches of the gold branched/corals nanostructure.

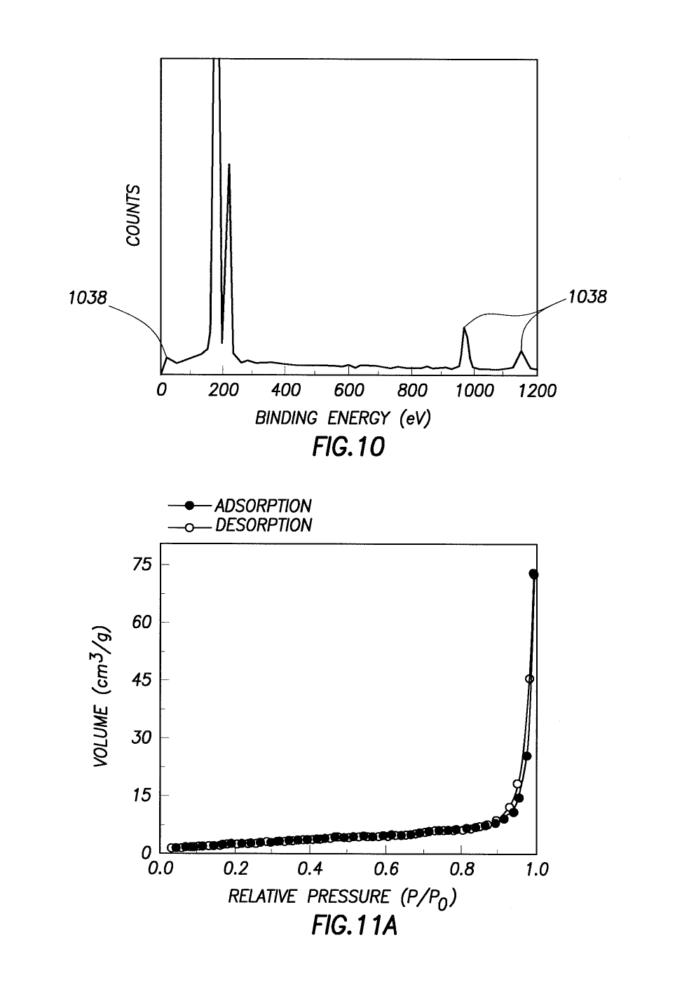

FIG. 10 shows the EDS measurements of the gold branched nanostructures in accordance with one or more embodiments of the invention. The EDS measurements may be performed to investigate the chemical composition and purity of the gold branched nanostructures. In addition, the peaks 1038 in the EDS measurements of gold branched nanostructures confirm the presence of Au atoms in the gold branched nanostructures.

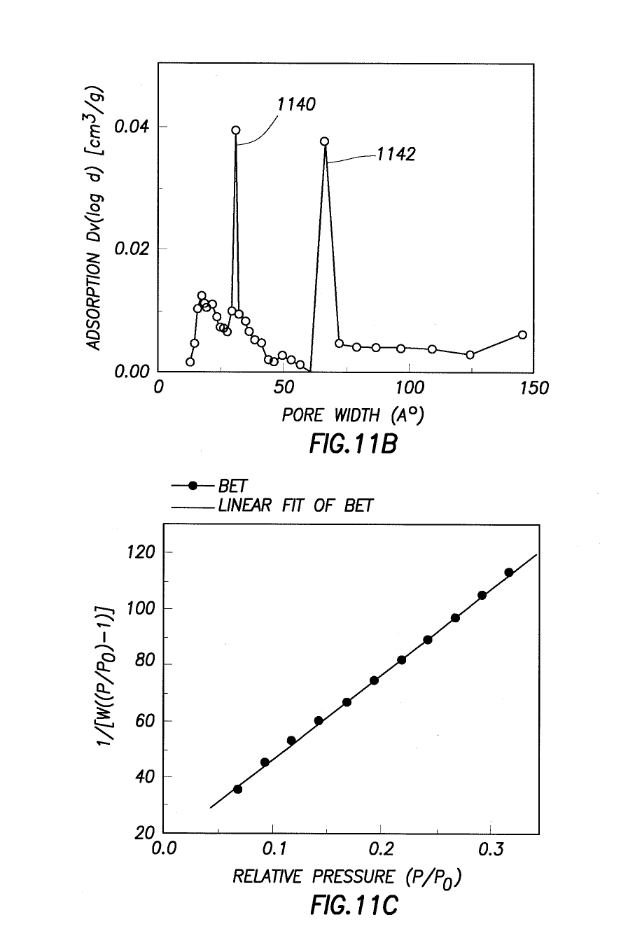

FIG. 11 shows a Brunauer-Emmett-Teller (BET) surface area and pore size distribution analysis of branches in accordance with one or more embodiments of the invention. The BET surface area and pore size may be performed to characterize the branched nanostructures. FIG. 11A presents the nitrogen adsorption-desorption isotherms of a gold corral sample calcinated at 150° C. for 8 hours. The isotherms may exhibit a type IV isotherm with a N₂ hysteresis loops in desorption branch as shown. As shown in FIG. 11A, the isotherms may be relatively flat in the low-pressure region (P/P₀<0.7). Also, the adsorption and desorption isotherms may be completely superposed, a fact which may demonstrate that the adsorption of the samples mostly likely occurs in the pores. At the relative high pressure region, the isotherms may form a loop due to the capillarity agglomeration phenomena. FIG. 11B presents a bimodal pore size distribution, showing the first peak 1140 at the pore diameter of 2.9 nm and the second peak 1142 at 6.5 nm. FIG. 11C shows the BET plots of gold branched nanostructures in accordance with one or more embodiments of the invention. A value of 10.84 m²/g was calculated for the specific surface area of branches in this example by using a multipoint BET-equation.

In one or more embodiments of the invention, the gold branched nanostructures dispersed in water may increase the nucleation sites for boiling, absorb electromagnetic energy, decrease the bubble lifetime due to high surface temperature and high porosity, and increase the interfacial turbulence by the water gradient temperature and the Brownian motion of the particles. The efficiency of a gold branched complex solution may be high because it may allow the entire fluid to be involved in the boiling process.

As demonstrated in the above figures and text, in accordance with one or more embodiments of the invention, the complex may include a number of different specific nanostructures chosen to maximize the absorption of the complex in a desired region of the electromagnetic spectrum. In addition, the complex may be suspended in different solvents, for example water or ethylene glycol. Also, the complex may be deposited onto a surface according to known techniques. For example, a molecular or polymer linker may be used to fix the complex to a surface, while allowing a solvent to be heated when exposed to the complex. The complex may also be embedded in a matrix or porous material. For example, the complex may be embedded in a polymer or porous matrix material formed to be inserted into a particular embodiment as described below. For example, the complex could be formed into a removable cartridge. As another example, a porous medium (e.g., fiberglass) may be embedded with the complex and placed in the interior of a vessel containing a fluid to be heated. The complex may also be formed into shapes in one or more embodiments described below in order to maximize the surface of the complex and, thus, maximize the absorption of EM radiation. In addition, the complex may be embedded in a packed column or coated onto rods inserted into one or more embodiments described below.

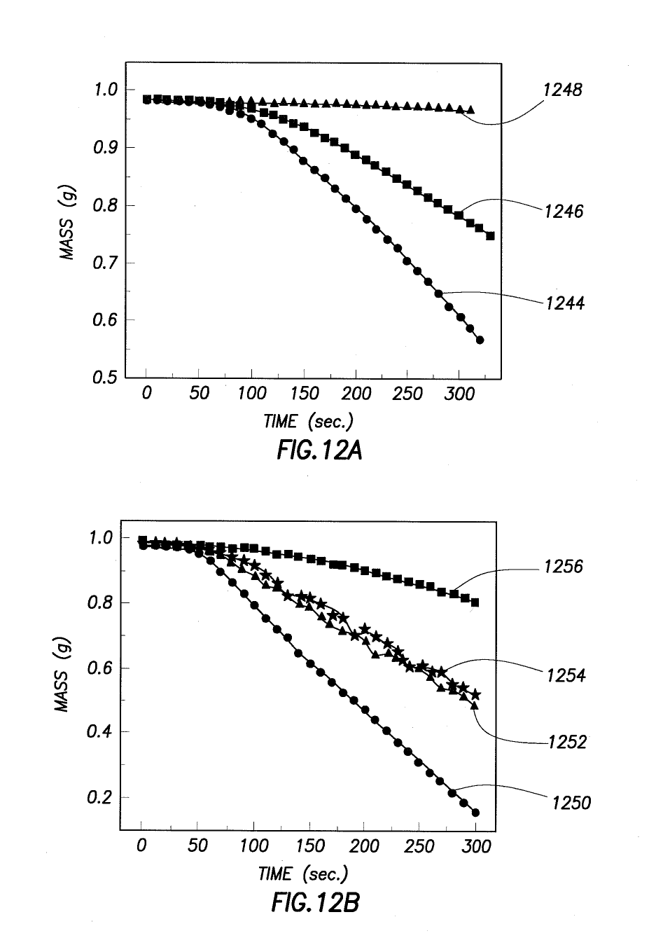

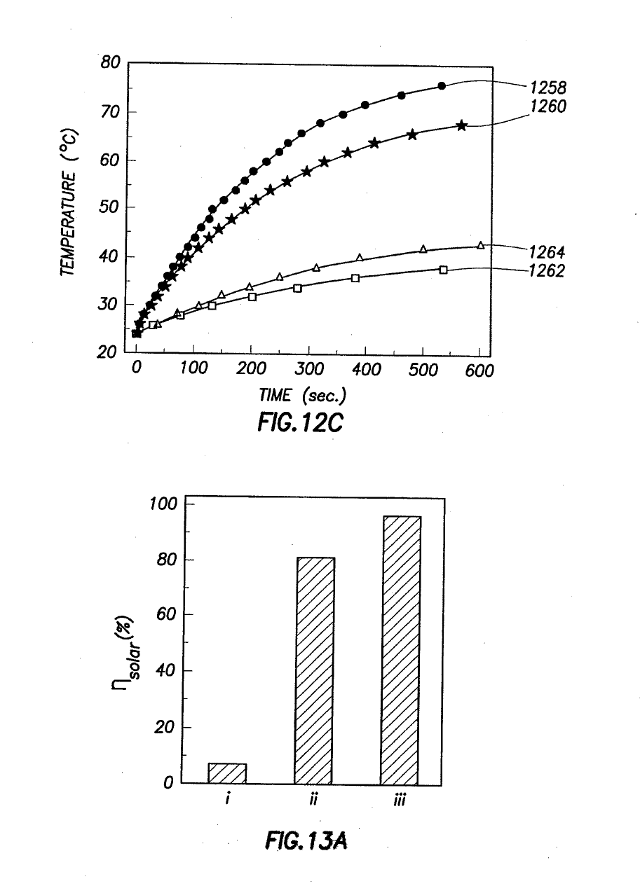

FIGS. 12A-12C show charts of the mass loss and temperature increase of different nanostructures that may be used in a complex in accordance with one or more embodiments of the invention. The results shown in FIGS. 12A-12C were performed to monitor the mass loss of an aqueous nanostructure solution for 10 minutes under sunlight (FIG. 12B) versus non-pulsed diode laser illumination at 808 nm (FIG. 12A). In FIG. 12A, the mass loss versus time of the laser illumination at 808 nm is shown for Eu₂O₃-coated nanoshells 1244, non-coated gold nanoshells 1246, and gold nanoparticles with a diameter of ˜100 nm 1248. Under laser exposure, as may be expected from the absorbance shown in FIG. 3, at 808 nm illumination, the coated and non-coated nanoshells exhibit a mass loss due to the absorbance of the incident electromagnetic radiation at 808 mm. In addition, as the absorbance is lower at 808 nm, the 100 nm diameter gold colloid exhibits little mass loss at 808 nm illumination. In FIG. 12A, the Au nanoparticles demonstrated a lower loss rate that was nearly the same as water because the laser wavelength was detuned from plasmon resonance frequency. The greatest mass loss was obtained by adding a layer around the gold nanoshells, where the particle absorption spectrum was approximately the same as the solar spectrum (see FIG. 3.)

In FIG. 12B, the mass loss as a function of time under exposure to the sun in accordance with one or more embodiments of the invention is shown. In FIG. 12B, the mass loss under sun exposure with an average power of 20 W is shown for Eu₂O₃-coated nanoshells 1250, non-coated gold nanoshells 1252, gold nanoparticles with a diameter of ˜100 nm 1254, and a water control 1256. As in the previous example, the greatest mass loss may be obtained by adding a rare earth or dielectric layer around a nanoshell.

The resulting mass loss curves in FIGS. 12A and 12B show significant water evaporation rates for Eu₂O₃-coated gold nanoshells. The mass loss may be slightly greater under solar radiation because the particles were able to absorb light from a broader range of wavelengths. In addition, the collective effect of aggregates broadens the absorption spectrum of the oxide-coated nanoparticles, which may help to further amplify the heating effect and create local areas of high temperature, or local hot spots. Aggregates may also allow a significant increase in boiling rates due to collective self organizing forces. The oxide layer may further enhance steam generation by increasing the surface area of the nanoparticle, thus providing more boiling nucleation sites per particle, while conserving the light-absorbing properties of the nanostructure.

FIG. 12C shows the temperature increase versus time under the 808 nm laser exposure in accordance with one or more embodiments of the invention. In FIG. 12C, the temperature increase under the 808 nm laser exposure is shown for Eu₂O₃-coated nanoshells 1258, non-coated gold nanoshells 1260, gold nanoparticles with a diameter of ˜100 nm 1262, and a water control 1264. As may be expected, the temperature of the solutions of the different nanostructures that may be included in the complex increases due to the absorption of the incident electromagnetic radiation of the specific nanostructure and the conversion of the absorbed electromagnetic radiation in to heat.

FIG. 13A is a chart of the solar trapping efficiency in accordance with one or more embodiments of the invention. To quantify the energy trapping efficiency of the complex, steam is generated in a flask and throttled through a symmetric convergent-divergent nozzle. The steam is then cooled and collected into an ice bath maintained at 0° C. The nozzle serves to isolate the high pressure in the boiler from the low pressure in the ice bath and may stabilize the steam flow. Accordingly, the steam is allowed to maintain a steady dynamic state for data acquisition purposes. In FIG. 13A, the solar energy capture efficiency (η) of water (i) and Eu2O3-coated nanoshells (ii) and gold branched (ii) nanostructures is shown. The resulting thermal efficiency of steam formation may be estimated at 80% for the coated nanoshell complex and 95% for a gold branched complex. By comparison, water has approximately 10% efficiency under the same conditions.

In one or more embodiments of the invention, the concentration of the complex may be modified to maximize the efficiency of the system. For example, in the case where the complex is in solution, the concentration of the different nanostructures that make up the complex for absorbing EM radiation may be modified to optimize the absorption and, thus, optimize the overall efficiency of the system. In the case where the complex is deposited on a surface, the surface coverage may be modified accordingly.

In FIG. 13B, the steam generation efficiency versus gold nanoshell concentration for solar and electrical heating in accordance with one or more embodiments of the invention is shown. The results show an enhancement in efficiency for both electrical 1366 and solar 1368 heating sources, confirming that the bubble nucleation rate increases with the concentration of complex. At high concentrations, the complex is likely to form small aggregates with small inter-structure gaps. These gaps may create “hot spots”, where the intensity of the electric field may be greatly enhanced, causing an increase in temperature of the surrounding water. The absorption enhancement under electrical energy 1366 is not as dramatic as that under solar power 1368 because the solar spectrum includes energetic photons in the NIR, visible and UV that are not present in the electric heater spectrum. At the higher concentrations, the steam generation efficiency begins to stabilize, indicating a saturation behavior. This may result from a shielding effect by the particles at the outermost regions of the flask, which may serve as a virtual blackbody around the particles in the bulk solution.

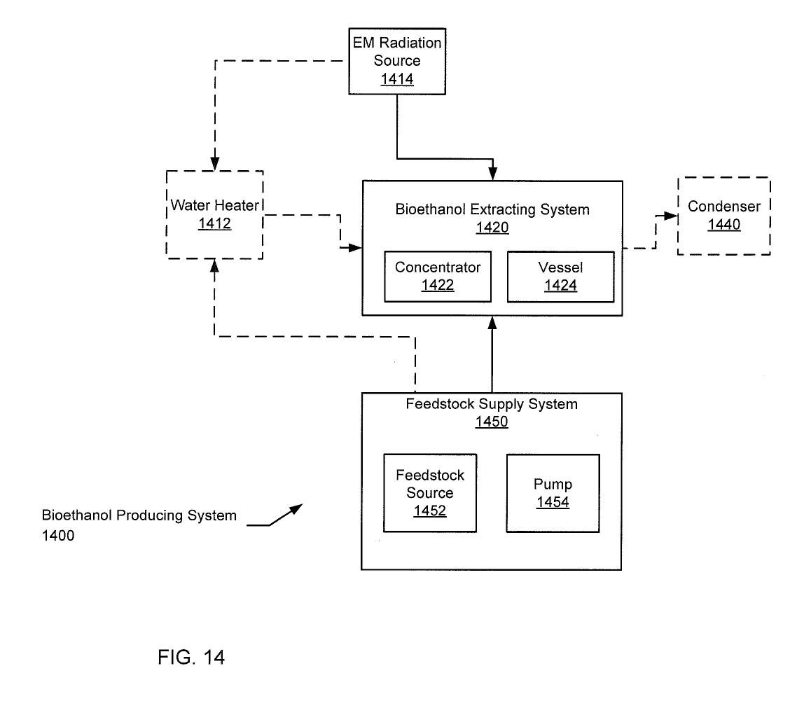

FIG. 14 shows a system in accordance with one or more embodiments of the invention. The bioethanol producing system 1400 demonstrated in FIG. 14 includes a bioethanol extracting system 1420. The bioethanol extracting system 1420 includes a vessel 1424 and concentrator 1422. The EM radiation source 1414 supplies the radiation to the concentrator 1422 to provide the energy to produce the bioethanol from the feedstock. The bioethanol producing system 1400 also includes a feedstock supply system 1450 that includes a feedstock source 1452 and pump 1454. The feedstock supply system 1450 may mix the feedstock into with a solution prior to pumping the feedstock solution into the vessel 1424. The bioethanol producing system 1400 may optionally include a water heater 1412 to preheat the feedstock solution in accordance with one or more embodiments of the invention. The bioethanol producing system 1400 may also include an optional condenser 1440 for collecting the bioethanol produced.

In one or more embodiments of the invention, each EM radiation source (e.g., EM radiation source 1414) is any other natural and/or manmade source capable of emitting one or more wavelengths of energy. The EM radiation source may also be a suitable combination of sources of EM radiation, whether emitting energy using the same wavelengths or different wavelengths.

Optionally, in one or more embodiments of the invention, each EM radiation concentrator (e.g., EM radiation concentrator 1422) is a device used to intensify the energy emitted by an EM radiation source. Examples of an EM radiation concentrator include, but are not limited to, one or more lenses (e.g., Fresnel lens, biconvex, negative meniscus, simple lenses, complex lenses), a parabolic trough, black paint, one or more disks, an array of multiple elements (e.g., lenses, disks), or any suitable combination thereof. An EM radiation concentrator may be used to increase the rate at which the EM radiation is absorbed by the complex.

In one or more embodiments of the invention, a vessel (e.g., vessel 1 1424) holds the feedstock solution and facilitates the transfer of energy (e.g., heat) to the feedstock solution to extract sugars present in the polysaccharides of the feedstock solution. A vessel may be designed and configured to operate under a pressure.

A vessel (e.g., vessel 1 1424), or a portion thereof, may include the complex. For example, a vessel may include a liquid solution (e.g., the feedstock, water, some other material, liquid or otherwise, such as ethylene glycol or glycine) that includes the complex, be coated on one or more inside surfaces with a coating of the complex, be coated on one or more outside surfaces with a coating of the complex, include a porous matrix into which the complex is embedded, include a packed column that includes packed, therein, a substrate on which the complex is attached, include rods or similar objects coated with the complex and submerged in the liquid solution, be constructed of a material that includes the complex, or any combination thereof. A vessel may also be adapted to facilitate one or more EM radiation concentrators (not shown), as described above.

A vessel may be of any size, material, shape, color, degree of translucence/transparency, or any other characteristic suitable for the operating temperatures and pressures to produce the amount and concentration of bioethanol. For example, a vessel may be a large, stainless steel cylindrical tank holding a quantity of solution that includes the complex and with a number of lenses (acting as EM radiation concentrators) along the lid and upper walls. In such a case, the solution may include the feedstock to be heated to extract the sugars from the feedstock and/or vaporize (distill) the bioethanol. Further, in such a case, the feedstock solution may include properties such that the complex remains in the solution when a filtering system (described below) is used. Alternatively, a chemical vessel may be a translucent pipe with the interior surfaces coated (either evenly or unevenly) with a substrate of the complex, where the pipe is positioned at the focal point of a parabolic trough (acting as an EM radiation concentrator) made of reflective metal.

Optionally, in one or more embodiments of the invention, a bioethanol extracting system 1420 may include one or more temperature gauges (not shown) to measure a temperature at different points inside a vessel and/or at other components of the bioethanol producing system 1400. For example, a temperature gauge may be placed at the point in a vessel where a vapor element exits the vessel (e.g., a vapor collector). Such temperature gauge may be operatively connected to a control system (not shown) used to control the amount and/or quality of vapor element produced in heating the feedstock solution. In one or more embodiments of the invention, a vessel may be pressurized where the pressure is read and/or controlled using a pressure gauge (not shown). Those skilled in the art will appreciate one or more control systems used to create heated fluid in heating the cool fluid may involve a number of devices, including but not limited to the temperature gauges, pressure gauges, pumps, agitators, fans, and valves, controlled (manually and/or automatically) according to a number of protocols and operating procedures. In one or more embodiments of the invention, the control system may be configured to maintain a maximum temperature (or range of temperatures) of a vessel so that the chemical mixture maintains (or does not exceed) a predetermined temperature.

Optionally, in one or more embodiments of the invention, one or more of the components of the bioethanol producing system 1400 may also include a filtering system (not shown). For example, a filtering system may be located inside a vessel and/or at some point before the chemical mixture enters the vessel. The filtering system may capture impurities (e.g., dirt and other solids) in the feedstock solution that may not be useful or that may inhibit the bioethanol production process. The filtering system may vary, depending on a number of factors, including but not limited to the configuration of the vessel, the configuration of the chemical mixture source, and the purity requirements of a vapor element. The filtering system may be integrated with a control system. For example, the filtering system may operate within a temperature range measured by one or more temperature gauges.

Optionally, in one or more embodiments of the invention, one or more pumps may be used in the bioethanol producing system 1400. A pump 1454 may be used to regulate the flow of the feedstock solution from the feedstock solution source 1452 into a vessel 1424 and/or the flow of the fluid element from a condenser (e.g., condenser 1 1440). A pump may operate manually or automatically (as with a control system, described above). Each pump may operate using a variable speed motor or a fixed speed motor. The flow of the feedstock solution, a vapor element from a vessel, and/or a fluid element from a condenser may also be controlled by gravity, a fan, pressure differential, some other suitable mechanism, or any combination thereof.

Optionally, in one or more embodiments of the invention, a storage tank of may be configured to store one or more fluid elements and/or vapor elements after the vapor element has been extracted from a vessel. In some embodiments of the invention, the storage tank may be a vessel or a vapor collector.

Optionally, in one or more embodiments of the invention, a supplemental water heater 1412 may be used in the bioethanol producing system 1400 to preheat the feedstock solution, or the solution used to make the feedstock solution.

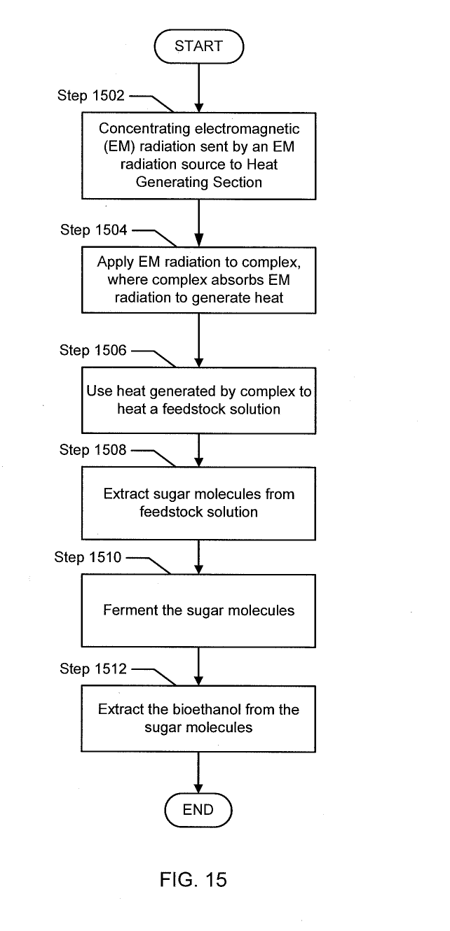

FIG. 15 shows a flowchart for a method for producing bioethanol in accordance with one or more embodiments of the invention. While the various steps in this flowchart are presented and described sequentially, one of ordinary skill will appreciate that some or all of the steps may be executed in different orders, may be combined or omitted, and some or all of the steps may be executed in parallel. Further, in one or more of the embodiments of the invention, one or more of the steps described below may be omitted, repeated, and/or performed in a different order. In addition, a person of ordinary skill in the art will appreciate that additional steps, omitted in FIG. 15, may be included in performing this method. Accordingly, the specific arrangement of steps shown in FIG. 15 should not be construed as limiting the scope of the invention.

One or more embodiments of the invention heat a feedstock solution to extract one or more sugars of the feedstock solution. The amount of feedstock solution that is heated by embodiments of the invention may range from a few ounces to thousands of gallons (or more) of feedstock solution.

Referring to FIG. 15, in Step 1502, EM radiation from an EM radiation source is concentrated and sent to the steam generating system. In Step 1504, the EM radiation irradiates a complex. The complex absorbs the EM radiation and generates heat. The heat is then used to heat a feedstock solution in Step 1506. The feedstock solution may have impurities (e.g., other elements and/or compounds) that are not needed or wanted when the fluid is in vapor form. The vessel containing the fluid may be any container capable of holding a volume of the fluid. For example, the vessel may be a pipe, a chamber, or some other suitable container. In one or more embodiments of the invention, the vessel is adapted to maintain its characteristics (e.g., form, properties) under high temperatures and pressures for extended periods of time. The complex may be part of a solution inside the vessel, a coating on the outside of the vessel, a coating on the inside of the vessel, integrated as part of the material of which the vessel is made, integrated with the vessel in some other way, or any suitable combination thereof. The fluid may be received in the vessel using a pump, a valve, a regulator, some other device to control the flow of the fluid, or any suitable combination thereof.

In one or more embodiments of the invention, the EM radiation is concentrated using an EM radiation concentrator, as described above with respect to FIG. 14. For example, the EM radiation may be concentrated using a lens or a parabolic trough. In one or more embodiments of the invention, the EM radiation is concentrated merely by exposing the vessel to the EM radiation.

In one or more embodiments of the invention, the complex absorbs the EM radiation to generate heat. The EM radiation may be applied to all or a portion of the complex located in the vessel. The EM radiation may also be applied to an intermediary, which in turn applies the EM radiation (either directly or indirectly, as through convection) to the complex. A control system using, for example, one or more temperature gauges, may regulate the amount of EM radiation applied to the complex, thus controlling the amount of heat generated by the complex at a given point in time. Power required for any component in the control system may be supplied by any of a number of external sources (e.g., a battery, a photovoltaic solar array, alternating current power, direct current power).

In Step 1508, sugar molecules are extracted from polysaccharides in the feedstock solution. In one or more embodiments of the invention, the heat generated by the complex is used to heat the feedstock solution to extract the sugars. In Step 1510, the sugar molecules extracted from the feedstock solution are fermented. The sugar molecules may be fermented using known techniques, for example through the addition of saccharomyces cerevisiae, a yeast. In Step 1512, the bioethanol is extracted. In one or more embodiments of the invention, the bioethanol may be extracted using the techniques described in PCT Application No. US2011/062497. After completing Step 1510, the process may end.

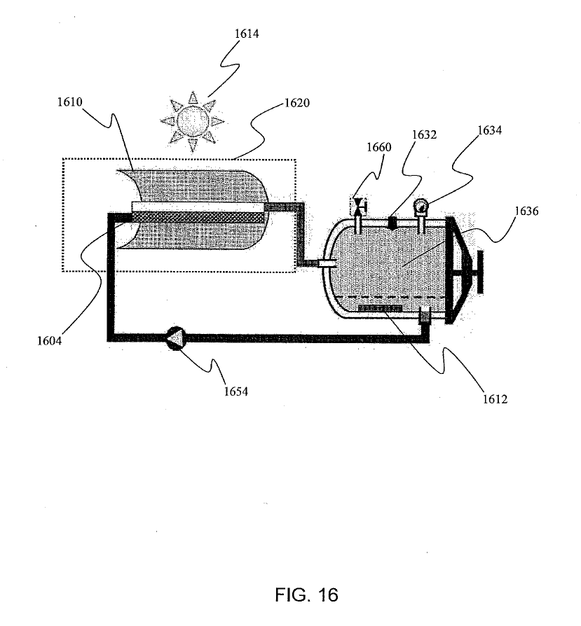

Consider the following example, shown in FIG. 16, which describes a system that produces steam used to heat the feedstock solution in accordance with one or more embodiments described above. This example is not intended to limit the scope of the invention. Turning to the example, the EM radiation source 1614 irradiates the complex 1604 through the use of the concentrator 1610 as part of the complex based bioethanol extracting system 1620. In this specific embodiment, the concentrator 1610 is parabolic mirror concentrating the EM radiation from the EM radiation source 1614 to a vessel containing the complex 1604. The complex based bioethanol extracting system 1620 may be used to supply steam to the chamber 1636. The chamber 1636 may include a temperature sensor 1632, a pressure sensor 1634, and a safety valve 1660. The chamber may also optionally include a heater 1612.

In one or more embodiments of the invention, steam is generated in the complex based bioethanol extracting system 1620. One of ordinary skill will appreciate that the chamber 1636 may include valves to isolate the chamber 1636 from the rest of the apparatus for the insertion or removal of the feedstock in the chamber 1636. At the conclusion of a cycle, a pump 1654 may be used to recycle the fluid for the next cycle. Alternatively, the pump 1654 may be used during the cycle to maintain the appropriate temperature and pressure necessary for heating the feedstock.

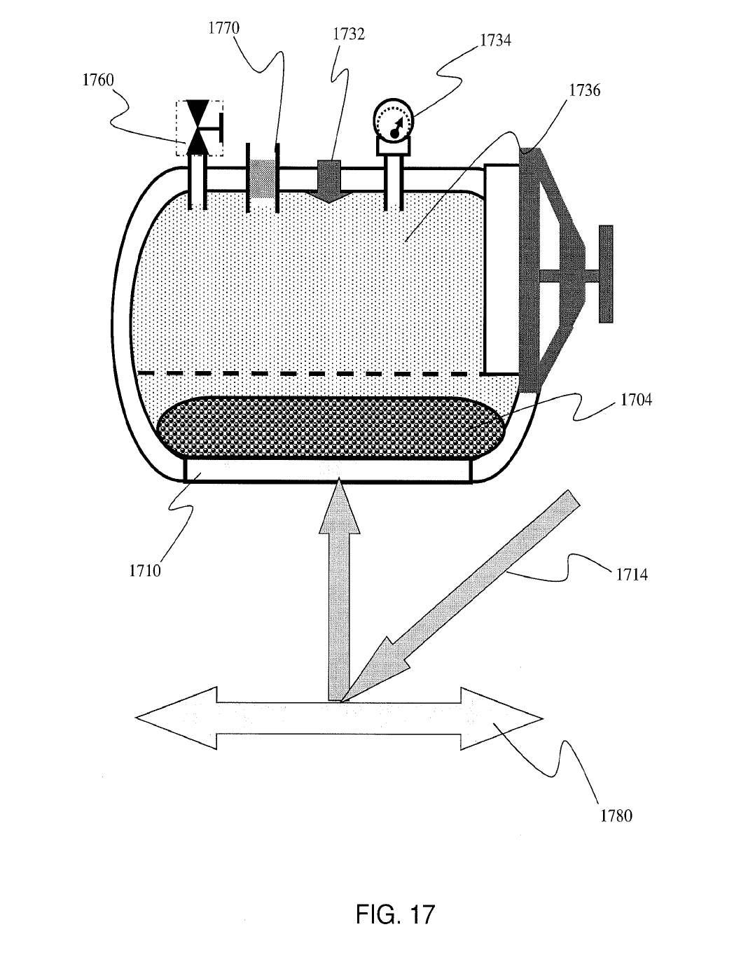

FIG. 17 illustrates an alternative configuration of the complex based bioethanol extracting system in accordance with one or more embodiments of the invention. The system shown in FIG. 17 includes a chamber 1736 with a temperature sensor 1732, a pressure sensor 1734, a supply valve 1770, and a safety valve 1760. The supply valve 1770 may be used to supply or maintain the supply of fluid in the chamber 1736. The complex 1704 may be disposed inside the chamber 1736, with the complex being accessible to EM radiation 1714, via the concentrator 1710. In one or more embodiments of the invention, the concentrator may be a lens or transparent material capable of handling the temperatures and pressures necessary to extract the sugar molecules from the feedstock within the chamber 1736. One or more embodiments of the invention may include an optical system 1780 designed to direct the EM radiation 1714 to the complex 1704, depending on the relative position of the EM radiation source. In one or more embodiments of the invention, such as that shown in FIG. 17, the system may be self-contained and portable.

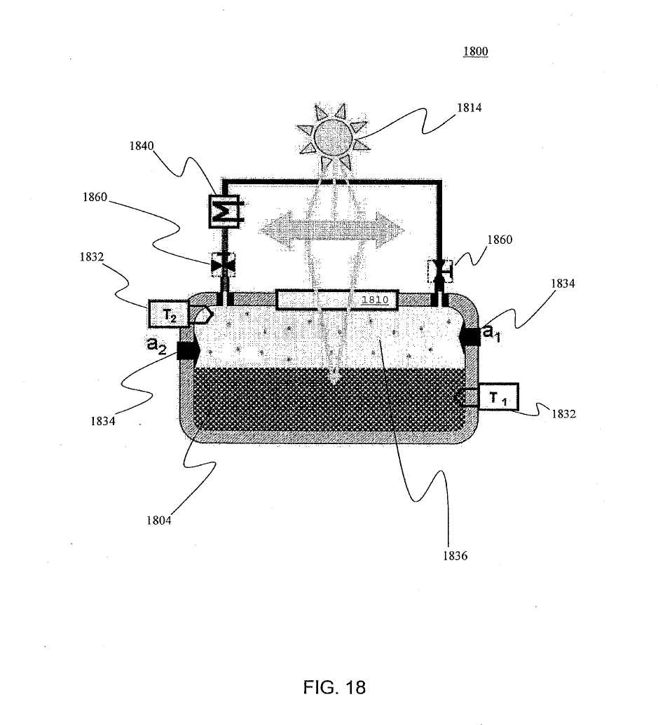

FIG. 18 illustrates a system for bioethanol extraction from feedstock in accordance with one or more embodiments of the invention. The system 1800 includes an EM radiation source 1814 that applies the radiation, via a concentrator 1810, to a complex 1804 located within the chamber 1836. The closed loop system 1800 may include one or more temperature sensors 1832, pressure sensors 1834, and safety valves 1860. The safety valves 1860 may open or close a loop containing a condenser 1840. During operation, feedstock, or feedstock solution, may be disposed inside the chamber 1836, at a position so as not to impede the EM radiation from the EM radiation source 1814 reaching the complex 1804. The EM radiation from the EM radiation source 1814 is absorbed by complex 1804. As a result of the irradiation, the complex 1804 generates heat in the chamber 1836 and, thus, increases the temperature of the fluid in the chamber 1836 and pressure in the chamber 1836. The fluid is converted to steam and may be applied to the feedstock to extract the sugar molecules and/or distill the bioethanol produced.

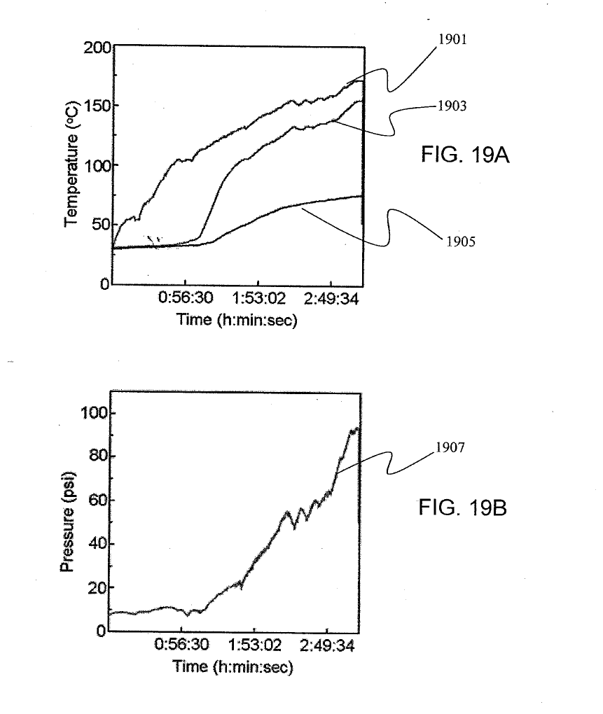

FIGS. 19A-19B illustrate the temperature and pressure that may be achieved in accordance with one or more embodiments of the invention. In FIG. 19A, the complex is a gold branched structure as described above in relation to FIGS. 8-11. The EM radiation source is the sun. In FIG. 19A, the safety relief valve begins to vent to the atmosphere when the solution inside the chamber reaches ˜170° C. and the pressure reaches ˜110 psi. In FIG. 19A, the temperature of the solution 1901 as a function of time indicates that the system may safely reach autoclave conditions. FIG. 19A also includes the temperature as a function of time before 1903 and after 1905 the condenser 1840. FIG. 19B is the pressure 1907 inside the chamber 1836 as a function of time. The irregularity of the pressure and temperature curves shown in FIGS. 19A and 19B are a result of clouds momentarily obstructing the sunlight which reduce the boiling intensity at different moments.

Embodiments of the claimed invention produce bioethanol from lignocellulosic biomass to generate fuel. The cell walls of grass contain cellulose and hemicellulose. These polysaccharides are degraded into smaller units in accordance with embodiments of the invention, which are accessible to yeast to perform fermentation.

Cellulose consists of D-Glucose units linked via a β-1,4 glycosidic bond. Hemicellulose contains Xylose, Mannose, Glucose and Galactose units. Glucose and Galactose are sugars which can be fermented by unmodified Saccharomyces cerevisiae, so their amount may be maximized in the pretreatment hydrolyzate.

The theoretically available amount of sugar in the feedstock may be deduced from a quantity of cellulose and hemicellulose. The amounts of hemicellulose and cellulose as well as other sugars available in feedstock are known. The amount of Glucose and Galactose available in Coastal hay may exceed the corresponding value for Alfalfa hay, which means Coastal may provide a more promising feedstock.

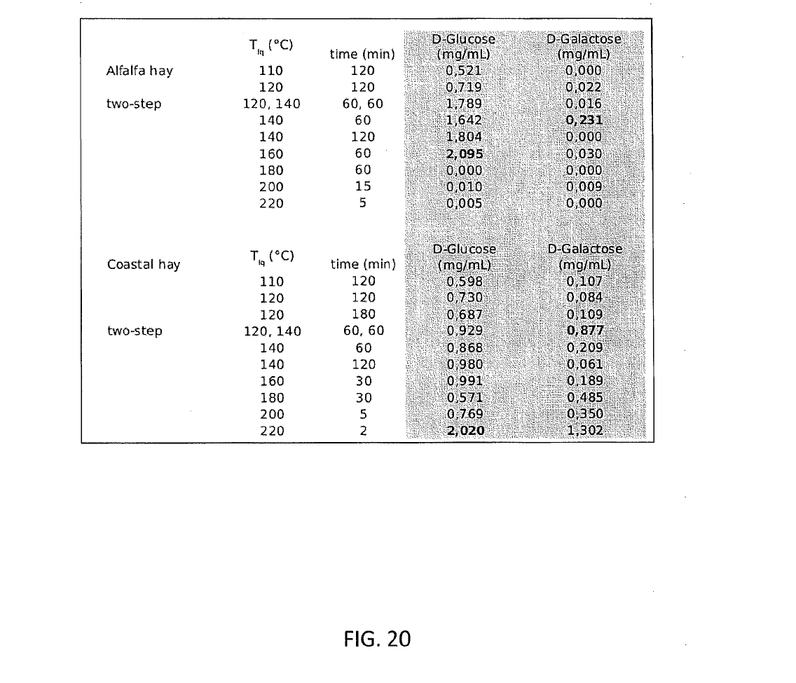

A pretreatment with pressurized hot water may be used to liberate sugars from polysaccharides. Referring to FIG. 20, several experiments with different temperatures and pretreatment times have been performed with a 5% w/v solid loading on a dry matter basis in accordance with embodiments of the invention. A Parr Instruments Model 4621 pressure vessel equipped with an electrical heating unit was used to determine the values in FIG. 20. The loaded vessel was heated to a certain temperature, which was kept constant for a defined time period. Thereafter the pressure was released whereby the temperature of the contents dropped rapidly. The sugar analysis of the pretreatment samples shown in FIG. 20 was carried out with a HPLC system.

In accordance with one or more embodiments of the invention, the temperature and pressure ranges shown in FIG. 19 may be used to liberate the sugars in a feedstock solution, as demonstrated in FIG. 20. In one or more embodiments of the invention, complex based distillation may be used to in the extraction of bioethanol.

In one or more embodiments of the invention, the complex based bioethanol production system may be a solar, portable system. For example, the complex based bioethanol production system may be used in the fields that provide the feedstock.

Embodiments of the invention may provide for bioethanol production without the use of caustic chemicals to break down plant cell walls, or the use of enzymes for that purpose. Embodiments may also provide for more economical production of bioethanol. Embodiments of the invention may provide an alternative to fossil-derived fuels.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method of producing bioethanol, the method comprising:
receiving, in a vessel comprising a complex, a feedstock solution comprising polysaccharides, wherein the complex is a least one selected from a group consisting of copper nanoparticles, copper oxide nanoparticles, nanoshells, nanorods, carbon moieties, encapsulated nanoshells, encapsulated nanoparticles, and branched nanostructures;
applying electromagnetic (EM) radiation to the complex, wherein the complex absorbs the EM radiation to generate heat;
extracting, using the heat generated by the complex, sugar molecules from the polysaccharides in the feedstock solution; and
fermenting the sugars molecules to generate bioethanol; and
extracting the bioethanol from the vessel.

2. The method of claim 1, wherein extracting the bioethanol from the vessel comprises:
condensing, using a condenser, the bioethanol from the vessel; and
storing the bioethanol in a storage tank.

3. The method of claim 1, wherein extracting the bioethanol from the vessel comprises:
applying additional EM radiation to the complex, wherein the complex absorbs the additional EM radiation to generate additional heat;
transforming, using the additional heat generated by the complex, the bioethanol to a vapor; and
extracting the vapor from the vessel.

4. The method of claim 1, further comprising:
concentrating the EM radiation applied to the vessel using a concentrator, wherein the concentrator is a lens integrated within a surface of the vessel.

5. The method of claim 1, wherein the complex is used in a manner selected from at least one of a group consisting of being coated on an interior of the vessel, being coated on the exterior of the vessel, integrated with material from which the vessel is constructed, embedded in a porous matrix, embedded with fiberglass and placed in the interior of the vessel containing the fluid, embedded on a substrate that is in a packed column, coated on rods at least partially submerged in the fluid, and suspended in the fluid in the vessel.

6. The method of claim 1, wherein the feedstock solution comprises at least one of a group Alfalfa, Coastal Bermudagrass, and switch grass.

7. The method of claim 1, wherein the sugar molecules are fermented using yeast.

8. The method of claim 7, wherein the yeast is saccharomyces cerevisiae.

9. The method of claim 1, wherein the vessel is pressurized.

10. A system for producing bioethanol, the system comprising:
a vessel comprising a complex and configured to:
receive, a feedstock solution comprising polysaccharides; and
enable electromagnetic (EM) radiation to be applied to the complex, wherein the complex absorbs the EM radiation to generate heat,
wherein the heat generated by the complex, is used to extract sugar molecules from the polysaccharides in the feedstock solution;
wherein the sugar molecules are fermented in the vessel to generate bioethanol,
wherein the complex is at least one selected from a group consisting of copper nanoparticles, copper oxide nanoparticles, nanoshells, nanorods, carbon moieties, encapsulated nanoshells, encapsulated nanoparticles, and branched nanostructures.

11. The system of claim 10, further comprising:
a vapor collector configured to collect the bioethanol; and
a condenser configured to receive the bioethanol from the vapor collector and condense the bioethanol.

12. The system of claim 10, further comprising:
an agitator configured to agitate the chemical mixture to assist extracting the sugars from the polysaccharides in the feedstock solution.

13. The system of claim 10, further comprising:
a control system adapted to control an amount of the bioethanol, wherein the control system comprises a first pump, a temperature gauge, and a pressure gauge.

14. The system of claim 10, wherein the first vessel comprises:
an EM radiation concentrator configured to intensify the EM radiation received from an EM radiation source.

15. The system of claim 10, wherein the EM radiation concentrator is one selected from a group consisting of a lens and a parabolic trough and wherein the vessel is a section of pipe coated with the complex.

16. The system of claim 10, wherein the complex is used in a manner selected from at least one of a group consisting of being coated on an interior of the vessel, being coated on the exterior of the vessel, integrated with material from which the vessel is constructed, embedded in a porous matrix, embedded with fiberglass and placed in the interior of the vessel containing the fluid, embedded on a substrate that is in a packed column, coated on rods at least partially submerged in the fluid, and suspended in the fluid in the vessel.

17. The system of claim 10, wherein the feedstock solution comprises at least one of a group Alfalfa, Coastal Bermudagrass, and switch grass;

18. The system of claim 10, wherein the sugar molecules are fermented using yeast.

19. The system of claim 18, wherein the yeast is saccharomyces cerevisiae.

20. The system of claim 1, wherein the vessel is pressurized.
